(19) United States
(12) Patent Application Publication
(10) Pub. No.: US 2011/0091939 A1
(43) Pub. Date: Apr. 21, 2011

(54) Methods and Compositions for Removing Specific Target Nucleic Acids

(76) Inventor: Longze Cui, San Diego, CA (US)

(21) Appl. No.: 12/907,791

(22) Filed: Oct. 19, 2010

Related U.S. Application Data
(60) Provisional application No. 61/253,394, filed on Oct. 20, 2009.

(51) Int. Cl. C12P 19/34; C07H 21/00

(52) U.S. Cl. 435/91.5; 536/25.4; 252/60

(57) ABSTRACT

The present invention provides methods, compositions and kits for removing specific target nucleic acid(s) from a nucleic acid sample. In particular, present invention provides a blocker oligonucleotide to prevent the target nucleic acid from binding to captor oligonucleotides, thus removing the target nucleic acid from a captor-binding nucleic acid pool. The present invention can be applied, for example, to remove high abundance mRNAs/cDNAs in high throughput nucleic acid sequencing.

### CROSS-REFERENCE TO RELATED APPLICATIONS

This application claims benefit of priority to U.S. provisional application Ser. No. 61/253,394, filed Oct. 20, 2009, the contents of which are incorporated by reference herein.

### BACKGROUND OF THE INVENTION

1. Field of the Invention

The present invention relates to methods and compositions for removing specific nucleic acids from a nucleic acid sample.

2. Description of the Related Art

DNA (deoxyribonucleic acid) microarrays have been the most important technology for large-scale studies of gene expression levels since the introduction of microarrays approximately 10 years ago. The microarray technology, which can simultaneously interrogate thousands of transcripts, enables us to obtain great achievements in a wide range of biological studies, including the identification of gene expression differences among diseased and healthy tissues, new insights into developmental processes and pharmacogenomic responses, and the evolution of gene regulation (Scherf et al. 2000; White 2001; Rifkin et al. 2003; Passador-Gurgel et al. 2007). However, microarray technology has several limitations. First, background levels of hybridization limit the accuracy of expression measurement of transcripts present in low abundance. Secondly, although gene expression differences among samples can be identified (Allison et al. 2006), hybridization results from a single sample may not provide a reliable measure of the relative expression of different transcripts because of the differences in hybridization properties and cross-hybridization among millions of microarray probes (Gautier et al. 2004). Finally, microarrays are limited to interrogating transcripts with relevant probes on the array.

Sequencing-based approaches may have the potential to overcome those limitations in measuring gene expression levels. New high throughput sequencing techniques enable hundreds of millions of short DNAs to be sequenced in several days. Various technologies, including those developed by 454 Life Sciences (Roche) (Margulies et al. 2005), IIlumina (formally Solexa) (Bennett et al. 2005), ABI (Tang et al. 2009) and Helicos (Harris et al. 2008), are currently available and have been used to investigate genetic variation (Korbel et al. 2007), transcription factor binding sites (Mikkelsen et al. 2007), and DNA methylation (Cokus et al. 2008). However, applications to the measurement of mRNA (messenger ribonucleic acid) expression levels have proceeded more slowly because of some limitations. First, it is relatively difficult to develop appropriate experimental protocols. Secondly, expression studies aim to identify (perhaps subtle) quantitative differences between samples, while other applications have, thus far, focused on detecting the absence or presence of an event, such as a transcription factor binding. Finally and most importantly, the cost is high because much more samples are needed when comparing expression levels across samples and identifying genes with differential expression.

One of the ideal ways to decrease the cost of measuring mRNA level with high throughput sequencing is multiplex, i.e. sequencing multiple samples. However, multiplex sequencing is limited by the maximum number of sequence tags that can be measured per run. A typical mammalian cell contains 10-30 pg total RNA. The majority of RNA molecules, however, are tRNAs and rRNAs. mRNA accounts for only 1-5% of the total cellular RNA depending on the cell type and physiological state. Approximately 360000 mRNA molecules are present in a single mammalian cell, with approximately 12000 different mRNA species per cell. Some mRNAs comprise as much as 3% of the mRNA pool whereas others account for less than 0.01%. These ‘rare’ or low abundance' mRNAs, which are important in special conditions, may have a copy number of only 1-15 molecules per cell (Alberts et al. 1994). Measurements of different transcripts using high throughput sequencing techniques (Mortazavi et al. 2008) (FIG. 3) showed that top 200 high abundance genes account for 66% of total sequencing tags and top 500 high abundance genes for around 80%. Therefore, if top 500 high abundance RNA species are reduced to low levels, the representation of non-high abundance genes in high throughput sequence tags can be increased by about 5 fold.

Several methods have been described (U.S. Pat. Pub. No. 20050123987A1, U.S. Pat. No. 6,391,592, U.S. Pat. Pub. No. 20060257902A1) to remove target nucleic acids for microarray analysis or construction of cDNA library. Nonetheless, such methods have shortcomings. For example, specific target RNA can be removed using RNase H digestion. After specific oligonucleotides hybridize with target RNA, RNA and DNA hybrid is digested using RNase H and the specific regions of the corresponding RNA are destroyed. Such RNase H treatment of RNA requires down-stream purification and thus is not a homogeneous process. This process also exposes the remaining sample RNA to potential damage of RNase H. These multiple steps and potential damage to non-target RNA limit its applications (U.S. Pat. Pub. No. 20050123987A1).

Another method uses non-extendable specific oligonucleotides, which are blocked at their 3′-prime end using special chemical linkages or non-extendable nucleotides (e.g. inverted T or dideoxy nucleotide terminators). These specialized 3′-blocked oligonucleotides can block reverse transcription from the corresponding RNA. This blocking method requires 3′-blocked primers to prevent them from serving as primers for initiating cDNA synthesis. However, if several hundreds of target RNAs are needed to be removed, several hundreds of specialized 3′-blocked primers are needed and it will increase its cost. In addition, this method cannot generate RNA pools without target RNAs (U.S. Pat. No. 6,391,592).

In another method, a target RNA is reverse transcribed into a target cDNA using an oligonucleotide primer specific to the target RNA. The remaining mRNAs are then reverse transcribed into T7-cDNAs with a T7 promoter sequence at the 5′-end and cRNA are transcribed from the T7-cDNAs using T7 RNA polymerase. Since target cDNA does not contain the T7 promoter sequence and can not be transcribed using T7 RNA polymerase, cRNA pools without the target cRNA can be obtained. Even though this method is simpler than block-aid method, there are still drawbacks. Many analysis such as high throughput sequencing, need mRNA or cDNA, not cRNA, to construct templates (libraries) before sequencing (U.S. Pat. Pub. No. 20060257902A1).

The use of biotinylated oligonucleotides specific for target mRNAs to capture target RNAs through streptavidin-coated magnetic beads is a simple method, but not practical because of costs, especially when the number of targets are increased or frequent changes in targets are needed. Using bridge oligonucleotides, target RNA can be indirectly captured, but its efficiency is low. If complete capture of target RNA is desired, the amount of bridge oligonucleotides should be much larger than that of target mRNAs and the amount of biotinylated capture oligonucleotides should be much larger than that of the bridge oligonucleotides, and the capacity of streptavidin-coated magnetic beads should be high enough to capture all of capture oligonucleotides with both of free and bridge oligonucleotides-captured (U.S. Pat. Pub. No.20060257902A1).

Currently several technologies for high-throughput sequencing such as array-based hybridization capture method (Okou et al. 2007), Hybrid Selection (Gnirke et al. 2009), Padlock Probes (Porreca et al. 2007) and Selector Probes (Dahl et al. 2007), were developed to capture target nucleic acids to be sequenced from nucleic acid pools. Few technologies have been developed for removing target nucleic acids from RNA samples for genome wide analysis using high throughput RNA/DNA sequencing.

As such, there is a continuing need in the art for technologies of removing target nucleic acids from nucleic acid pools for high throughput sequencing analysis. The present invention provides such a technology to satisfy this need and provides other benefits as well.

### SUMMARY OF THE INVENTION

The present invention pertains to methods, compositions and kits for removing specific target nucleic acids from a nucleic acid pool. The methods can be used to make nucleic acid samples desirable for high throughput nucleic acid sequencing analysis.

In some embodiments, present invention provides a method of obtaining a nucleic acid pool with removal of a target nucleic acid from a nucleic acid sample, comprising: A, contacting the nucleic acid sample with a blocker oligonucleotide, wherein the blocker oligonucleotide comprises a specific region, which has a sequence complementary to a specific sequence of the target nucleic acid, and an oligo(dT/dA) region (e.g. the oligo(dT) and the oligo(dA) region are used for removing target nucleic acids from RNA and cDNA samples, respectively); B, contacting the nucleic acid sample with captor oligonucleotides, wherein the captor oligonucleotides comprise a specific region, which has several random nucleotides, and an oligo(dT/dA) region; C, separating a captor-binding nucleic acid pool from a nucleic acid pool that does not hybridize to the captor oligonucleotides, therefore obtaining a nucleic acid pool with no or reduced amount of the target nucleic acid. The nucleic acid sample, for example, may be a total RNA, an mRNA or a cDNA sample.

In particular embodiment of the invention, the blocker oligonucleotide comprises a specific region, which has a sequence complementary to a specific sequence that is adjacent to the 5′-end of poly(A) region of the target nucleic acid, and an oligo(dT/dA) region, wherein the blocker oligonucleotide hybridizes stronger to the target nucleic acid than the captor oligonucleotides. The blocker oligonucleotide, for example, may be complementary to a junction region of the target nucleic acid including the 5′ poly(A) region and the adjacent upstream specific sequence. In some embodiments of the invention, the blocker oligonucleotide comprises a peptide nucleic acid (PNA), a locked nucleic acid (LNA) or a 3′ modified oligonucleotide (e.g. dideoxynucleotide) that is complementary to a specific sequence of the target nucleic acid. In other embodiment of the invention, the blocker oligonucleotide comprises an oligo(dT/dA) region that is equal or longer than the oligo(dT/dA) region of the captor oligonucleotides. In some embodiments of the invention, the specific region of the blocker oligonucleotide (e.g. at least 5, 6, 7, 8, 9, 10, 11, 12, 13, 14, 15, 16, 17, 18, 19, 20, 21, 22, 23, 24, 25 bases or longer) is longer than the specific region of the captor oligonucleotides. In some embodiments of the invention, the specific region of the captor oligonucleotides has at least 1, 2, 3, 4, 5, 6, or more random nucleotides.

In some embodiments of the invention, a plurality of blocker oligonucleotides are used to hybridize to a plurality of target nucleic acids, blocking them from binding to captor oligonucleotides. The method can be applied to make a nucleic acid pool with removal of a plurality of target nucleic acids. In some embodiments of the invention, the blocker oligonucleotides are designed to be complementary to specific sequences of high abundance nucleic acids in a transcriptome. The method can be applied to remove a plurality of high abundance nucleic acids from a nucleic acid sample. In the high throughput sequence analysis, the abundance of gene expression is represented by the sequence tag number. The blocker oligonucleotides can, for example, be designed to be complementary to top genes with highest expression ranking in a transcriptome (e.g. top 1-50, 50-200, or 200-500 ranking genes). In particular embodiments of the invention, the blocker oligonucleotides are designed to be complementary to top 50, 200, or 500 genes with highest expression ranking in a transcriptome.

In some embodiments of the invention, the captor oligonucleotides are linked to a capture structure, which allows the captor oligonucleotides and any specifically bound nucleic acids to be separated from other nucleic acid populations. In particular embodiments of the invention, the captor oligonucleotides are linked to a biotin. And the captor-binding nucleic acid pool can be separated by binding to immobilized streptavidin or avidin (e.g. streptavidin-coated magnetic beads or avidin-coated magnetic beads).

In some embodiments, the present invention further provides methods for retrieving the target nucleic acids. For example, after removing captor-binding nucleic acids from the nucleic acid sample, the target nucleic acids with poly(A) sequence can bind to a poly(dT) column and be eluted from the poly(dT) column. In some embodiments, the blocker oligonucleotide is linked to a capture structure which allows the blocker oligonucleotide to be separated from the rest of the nucleic acid population. The capture structure of the blocker oligonucleotide is different from that of captor oligonucleotides. In some embodiments, the blocker oligonucleotide can be linked to a poly(C) or poly(G) sequence, which allows the blocker oligonucleotide to be captured by biotinylated poly(G) or poly(C) nucleic acids, respectively. In another embodiment, after captor-binding RNAs are removed from the nucleic acid sample, cDNAs of the target nucleic acids can be generated by reverse transcription using the blocker oligonucleotides as primers, and the cDNAs can be later separated from the RNA/DNA mixture using RNase H digestion.

In some embodiments, the present invention provides a method for determining the optimal captor oligonucleotides that selectively bind to unblocked mRNAs, comprising: A, making captor oligonucleotides with different number of random nucleotides (e.g. 1, 2, 3, 4, 5, 6 or more random nucleotides) and an oligo(dT/dA) sequence; B, contacting a blocker oligonucleotide specific to an abundant target gene and the captor oligonucleotides to a nucleic acid sample; C, separating the captor-binding nucleic acid pool from the non-captor-binding nucleic acid pool; D, determine the amount of the target gene and an unblocked gene in the captor-binding nucleic acid pool (blocked pool) and the non-captor-binding nucleic acid pool (unblocked pool); E, calculate the ratio of the target gene in the blocked pool vs. unblocked pool (Tᵣₐₜᵢₒ), and the ratio of an unblocked gene in the blocked pool vs. unblocked pool (Uᵣₐₜᵢₒ); F, compare the ratio (Finalᵣₐₜᵢₒ) between Tᵣₐₜᵢₒ and Uᵣₐₜᵢₒ for different captor oligonucleotides, wherein the captor oligonucleotide with the lowest Finalᵣₐₜᵢₒ is the optimal captor oligonucleotide.

In some embodiments, present invention provides a composition, comprising: A, a blocker oligonucleotide, wherein the blocker oligonucleotide comprises a specific region, which has a sequence complementary to a specific sequence of a target nucleic acid, and an oligo(dT/dA) region; B, captor oligonucleotides, wherein the captor oligonucleotides comprise a specific region, which has several random nucleotides, and an oligo(dT/dA) region. In particular embodiments, the composition of present invention comprises a blocker oligonucleotide that comprises a specific region, which has a sequence complementary to a specific sequence that is adjacent to the 5′-end of poly(A) region of the target nucleic acid. In some embodiments, the composition of present invention comprises blocker oligonucleotides that comprise specific regions, which have sequences complementary to top genes with the highest expression ranking in a transcriptome (e.g. top 1-50, 50-200, or 200-500 ranking genes). In some embodiments, the composition comprises captor oligonucleotides that are linked to a capture structure, which allows the captor oligonucleotides and any specifically bound nucleic acids to be separated from other nucleic acid population. In particular embodiments, the composition comprises captor oligonucleotides linked to biotin. In additional embodiments, composition further comprises immobilized streptavidin or avidin (e.g. streptavidin-coated or avidin-coated beads).

In some embodiments, the present invention provides a kit for removing a target nucleic acid from a nucleic acid pool, comprising: A, a blocker oligonucleotide, wherein the blocker oligonucleotide comprises a specific region, which has a sequence complementary to a specific sequence of the target nucleic acid, and an oligo(dT/dA) region; B, captor oligonucleotides, wherein the captor oligonucleotides comprise a specific region, which has several random nucleotides and an oligo(dT/dA) region.

### BRIEF DESCRIPTION OF THE DRAWINGS

FIG. 1. Strategy of Obtaining an mRNA Pool with Specific Target mRNA Removed

First, a blocker oligonucleotide hybridizes with a target mRNA at the junction region (a region including the 5′ poly(A) region and the adjacent specific sequence) and blocks other oligonucleotides from further hybridizing with the target mRNA at the junction region. Secondly, biotinylated captor oligonucleotides hybridize with unblocked mRNAs at the junction region. Thirdly, the unblocked mRNAs are captured to streptavidin-coated magnetic beads through biotinylated captor oligonucleotides. Finally, the captured mRNA pool is released from magnetic beads. Alternatively, cDNA was generated by reverse transcription during the capturing step, and both mRNA and cDNA pools without the target nucleic acid can be captured to streptavidin beads.

FIG. 2. Removal of GADPH mRNA from an mRNA Pool

The PCR products of GAPDH (blocked gene) and KLK3 (unblocked gene) in unblocked pool (supernatant) vs. blocked pool (streptavidin-coated magnetic beads captured) were compared after blocking with different concentrations of a GAPDH blocker oligonucleotide and captured by biotinylated captor oligonucleotides and streptavidin beads. Note that most of GAPDH was left in the supernatant and much less was present on the beads. On the contrary, most of KLK3 was enriched on the beads and much less was left in the supernatant.

FIG. 3. Accumulated Distribution of Top 500 Genes with Highest Sequence Tag Number Ranking in High Throughput Sequencing Analysis (Mouse).

Accumulated distribution of top 500 genes with highest sequence tag number ranking among the whole genome was shown (Adapted from Mortazavi et al. 2008). Top 50 genes account for around 50% of the total sequence tags, top 200 genes for around 60% and top 500 genes for around 80% of the total sequence tags.

### DETAILED DESCRIPTION

The present invention pertains to methods, compositions and kits for removing specific target nucleic acids from a nucleic acid pool. The methods can be used to make nucleic acid samples desirable for high throughput nucleic acid sequencing analysis.

Global transcriptome analysis plays an important role in understanding how altered expression of genetic variants contributes to complex diseases such as cancer, diabetes, and heart disease. Analysis of genome-wide differential RNA expression provides researchers with greater insights into biological pathways and molecular mechanisms that regulate cell fate, development, and disease progression.

Even though microarray revolutionized biological research by enabling gene expression comparisons on a transcriptome-wide scale, it has limitations. RNA-Seq, the use of high throughput sequencing technologies to retrieve information about RNA contents in a sample, can overcome some limitations of microarray in gene expression analysis. High throughput sequencing technologies generate millions of short sequence reads/tags from nucleic acid samples, and calculate the expression level of a gene based on its sequence tag number. RNA-Seq can generate reproducible data with few systematic differences among technical replicates and discover novel transcripts and splicing. Statistically, in evaluation of the variation across technical replicates only a small proportion (˜0.5%) of genes showed clear deviations. In addition, the RNA-Seq could identify 30% more differentially expressed genes than microarray at the same false discovery rate and provide better estimates of absolute transcript levels (Marioni et al. 2008, Fu et al. 2009). However, applications of RNA-Seq to the measurement of mRNA expression levels have proceeded more slowly than expected because of the cost, which is the major hurdle.

The best way to reduce the cost may be multiplex, i.e. sequencing multiple samples simultaneously. For example, 8 samples can be sequenced per run using Illumina GA II. In fact, it is possible to load multiple samples in one lane. However, multiplexing in one lane is not currently practical for analyzing low abundance RNAs because the number of sequence tags generated per lane is limited and small proportion of genes with high abundance mRNAs account for more than half of sequence tags. If multiplex is desired, high abundance mRNAs must be removed from RNA pools or reduced to low levels. The RNA-Seq data (Mortazavi at al. 2008) (FIG. 3) showed that top 200 high abundance genes account for 66% of total sequencing tags and top 500 high abundance genes for around 80%. Therefore, if top 500 high abundance RNA species are reduced to low levels, the representation of low abundance genes in high throughput sequence tags can be increased by about 5 fold.

The present invention provides a simple, cost-effective and flexible method to reduce high abundance target nucleic acids in a sample, and increase the efficiency of RNA-Seq in detecting low abundance gene products in transcriptome research. It applies not only to RNA-Seq, but also to other fields. For example, Hybrid Selection, a hybridization-based technology, has a bias in capturing targets because of the big difference in hybridization property, resulting in some targets much more enriched than others. The present invention can be applied to reduce the amount of highly enriched targets to low levels in Hybrid Selection Analysis, resulting in better and unbiased representation of other genes as in the transcriptome analysis.

The terms “target”, “target nucleic acid”, “blocked nucleic acid” used interchangeably herein, refer to an undesirable mRNA or cDNA, the removal of which makes the resulting mRNA/cDNA pool more desirable and suitable for further analysis such as a high throughput sequencing analysis. The target nucleic acid may be, for example, high abundance mRNA or cDNA, the removal of which can result in improved sensitivity and reduced variability of measuring low abundance mRNA in the high throughput sequencing analysis.

The term “nucleotide/nucleoside analog” used herein, refer to a natural or unnatural variant of a nucleotide/nucleoside. These analogs share some common structural features with naturally occurring nucleotides/nucleosides such that when incorporated into a nucleic acid sequence, they allow hybridization to a complementary natural nucleic acid sequence. For example, nucleotide analogs include inosine, dideoxynucleotide, and LNA nucleotides. Nucleotide analogs also include modified nucleotides such as methylated nucleotides. The term “nucleotide/nucleoside” used herein, generally includes a naturally occurring nucleotide/nucleoside and its analogs.

The term “nucleic acid” used interchangeably herein with “polynucleotide” and “oligonucleotide” refers to a polymeric form of nucleotides at any length, which comprises purine or prymidine bases, or other naturally or unnaturally modified or derivatized nucleotide bases. A nucleic acid may comprise naturally occurring nucleotides and nucleotide analogs as described herein. The backbone of a nucleic acid may comprise sugar and phosphate group, as may typically be found in RNA and DNA, or modified or substituted sugar and phosphate group. A nucleic acid used herein may be, for example, an RNA, a DNA, a LNA, or a peptide nucleic acid (PNA), where a pseudo-peptide backbone replaces the ribose phosphodiester backbone in RNA/DNA.

The terms “blocker” and “blocker oligonucleotide” used interchangeably herein, refer to an oligonucleotide that can hybridize to a specific sequence of a target nucleic acid and prevent other oligonucleotides from binding to the same sequence. The blocker oligonucleotide includes naturally occurring nucleic acid and modified or derivatized nucleic acids as described above. A blocker oligonucleotide may be, for example, an RNA, a DNA, a LNA, or a PNA.

The blocker oligonucleotide has a specific region complementary to a specific sequence of a target nucleic acid, and an homogenous oligo(dT/dA) region. When the target nucleic acid is an mRNA, the specific region of the blocker oligonucleotide may be complementary to a specific sequence adjacent to the 5′-end of poly(A) tail of the target nucleic acid. The term, “poly(A) tail/sequence/region of a target nucleic acid” used herein refers to the poly(A) region located at the 3′ end of a mRNA. The corresponding sequence in a cDNA (the anti-sense strand) is the poly(dT) region located at the 5′ end of the cDNA sequence. In cases where “poly(A) tail of a target nucleic acid” is used, the same design principal and concept is applied to the corresponding poly(dT) region of a cDNA sequence. The term “adjacent to the 5′-end of poly(A) tail” as used herein, refers to the fact that said specific sequence of the target nucleic acid locates close to the 5′-end of poly(A) tail. The first 3′-end nucleotide of said specific sequence may locate several bases upstream to the poly(A) tail. For example, it may locate at −1, −2, −3, −4, or −5 upstream position in relation to the poly(A) tail, wherein −1, −2, −3, −4, and −5 position indicates the first, second, third, forth, and fifth base upstream to the 5′-end of poly(A) tail, respectively. The term “the junction region” of a nucleic acid used herein, refers to a region encompassing the 5′ poly(A) region and its adjacent upstream specific sequence of the target mRNA. The specific region of a blocker oligonucleotide of a target mRNA is designed to be complementary to a part or all of the junction region of the target mRNA. The oligo(dT/dA) region of a blocker oligonucleotide is designed to be complementary to the poly(A) or the equivalent region of a target nucleic acid. For mRNA target sequences, the oligo(dT/dA) region comprises a homogenous sequence of nucleotides with thymine (T) or Uracil (U) bases. For cDNA target sequences, the oligo(dT/dA) region correspondingly comprises a nucleic acid sequence with Adenine (A) bases. When the target nucleic acid is a cDNA, the junction region of the cDNA encompasses the 3′ end of the poly(dT) region and its adjacent downstream specific sequence of the target cDNA. A blocker oligonucleotide is correspondingly designed to be complementary to this junction region of the target cDNA.

The blocker oligonucleotide may be modified to prevent extension by reverse transcription or PCR. For example, the blocker can be a PNA, which can not be recognized by DNA polymerase or reverse transcriptase, thus preventing extension of the blocker nucleotide in PCR or reverse transcription reactions. The 3′ end of the blocker can also incorporate non-extendable nucleotides analog such as dideoxynucleotide to prevent further extension. The blocker oligonucleotide may also be linked to or incorporated with a capture structure, thus allowing the target nucleic acid to be captured by various methods. For example, capture structure-linked blocker oligonucleotide may be separated by physical means, electromagnetic means, ligand-receptor binding, antigen-antibody association, or complementary nucleic acid pairing. The capture structure may be magnetic beads that allow capturing blocker oligonucleotides in a magnetic field. The capture structure may be a poly(C) nucleic acid sequence that can be separated using biotinylated oligo(G).

The terms “oligo(dT) or oligo(dA) region” or “oligo(dT/dA) region” used herein, refer to a homogenous sequence having the same nucleotide repeated across the length such as a repeat of dT or dA residues. Oligo(dT/dA) region may comprise an RNA, a DNA, a LNA or a PNA. Oligo(dT) region may comprise a repeated sequence of Thymine or Uracil ribonucleotide, deoxyribonucleotide or its analogs. Oligo (dA) region may comprise a repeated sequence of Adenine ribonucleotide, Adenine deoxyribonucleotide or its analogs. The number of nucleotides in an oligo(dT/dA) region is, for example, at least 5, 6, 7, 8, 9, 10, 11, 12, 13, 14, 15, 16, 17, 18, 19, 20 or more.

The term “complementary” used herein, refers to the capability of a nucleic acid to hybridize to another nucleic acid strand or duplex even if less than all the nucleobases base-pair with counterpart nucleobases. For example, a “complementary” nucleic acid comprises a sequence with at least 70%, 75%, 80%, 85%, 90%, 95%, 96%, 97%, 98%, 99%, 100%, and any range derivable therein, of the nucleotide sequence capable of base-pairing with a single or double stranded nucleic acid molecule during hybridization.

The term “captors” and “captor oligonucleotides” used interchangeably herein, refer to a pool of oligonucleotides that have an oligo(dT/dA) region and a specific region with several random nucleotides. The specific region of captor oligonucleotides encompasses all the possible sequence combinations of the random nucleotides, which allows the captor oligonucleotides to hybridize with any nucleic acid at said specific region. With both the oligo(dT/dA) region and the specific region, captor oligonucleotides can hybridize to the junction region of any mRNA/cDNA in a sample. The number of the random nucleotides in the specific region of captor oligonucleotides can be, for example, 1, 2, 3, 4, 5, 6 or more. The optimal length of the random nucleotides in the specific region of captors can be determined empirically. In the case of RNA samples, captor oligonucleotides comprise a oligo(dT) region and several random nucleotides. In the case of cDNA samples, captor oligonucleotides comprise a oligo(dA) region and several random nucleotides.

In some embodiments, present invention provides a method of obtaining a mRNA pool with removal of a target nucleic acid from a nucleic acid sample, comprising: A, contacting the nucleic acid sample with a blocker oligonucleotide, wherein the blocker oligonucleotide comprises a specific region, which has a sequence complementary to a specific sequence of the target nucleic acid, and an oligo(dT/dA) region; B, contacting the nucleic acid sample with captor oligonucleotides, wherein the captor oligonucleotides comprise a specific region, which has several random nucleotides, and an oligo(dT/dA) region; C, separating a captor-binding nucleic acid pool from nucleic acids that do not hybridize to the captor oligonucleotides, therefore obtaining a nucleic acid pool with no or reduced amount of the target nucleic acid. The method of present invention concerns isolation of a nucleic acid pool with a poly(A) or poly(dT) sequence such as mRNA, cDNA pool. The nucleic acid sample, for example, may be a total RNA sample, an mRNA sample or a cDNA sample.

In particular embodiments of the invention, the blocker oligonucleotide comprises a specific region, which has a sequence complementary to a specific sequence that is adjacent to the 5′-end of poly(A) region of the target nucleic acid, and an oligo(dT/dA) region, wherein the blocker oligonucleotide hybridizes stronger to the target nucleic acid than the captor oligonucleotides. The blocker oligonucleotide, for example, may be complementary to a junction region of the target nucleic acid sequence including the 5′ poly(A) region and the adjacent upstream specific sequence of the target nucleic acid.

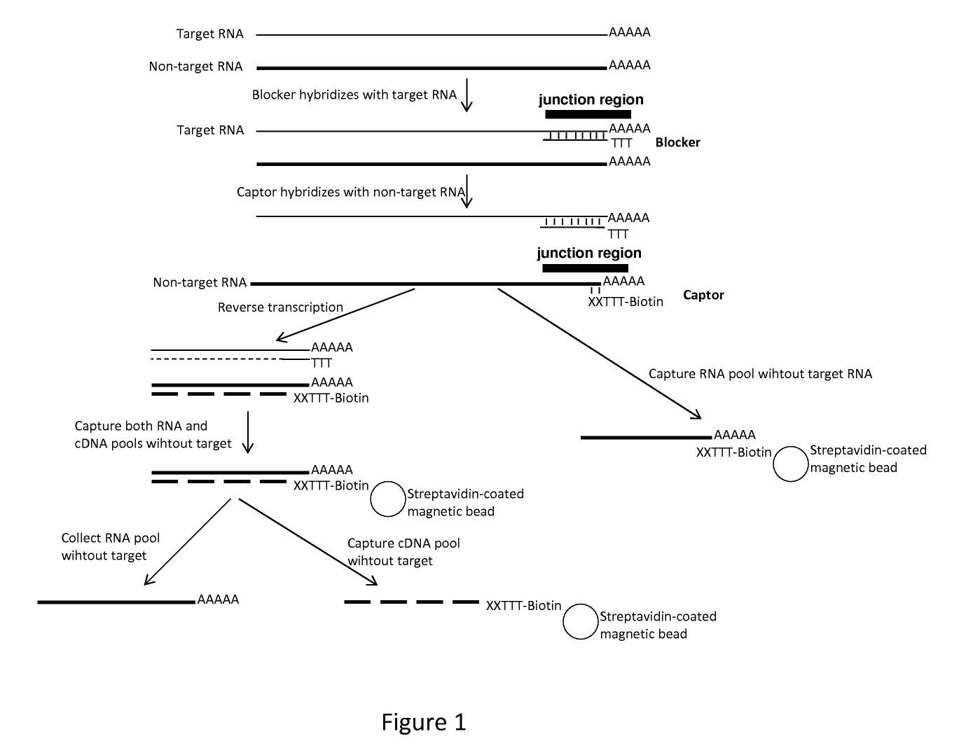

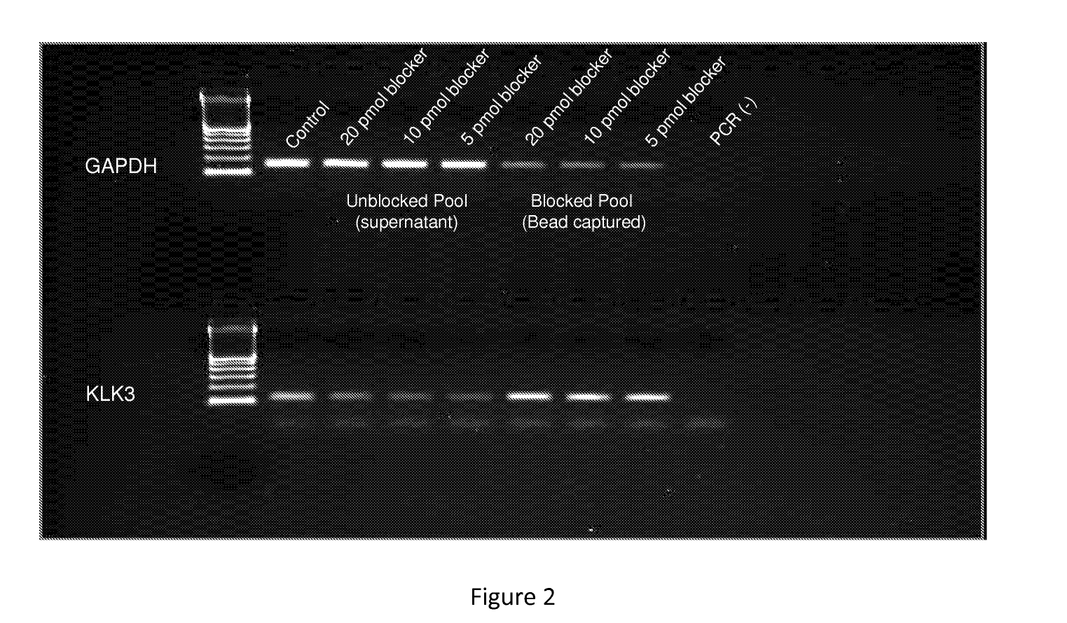

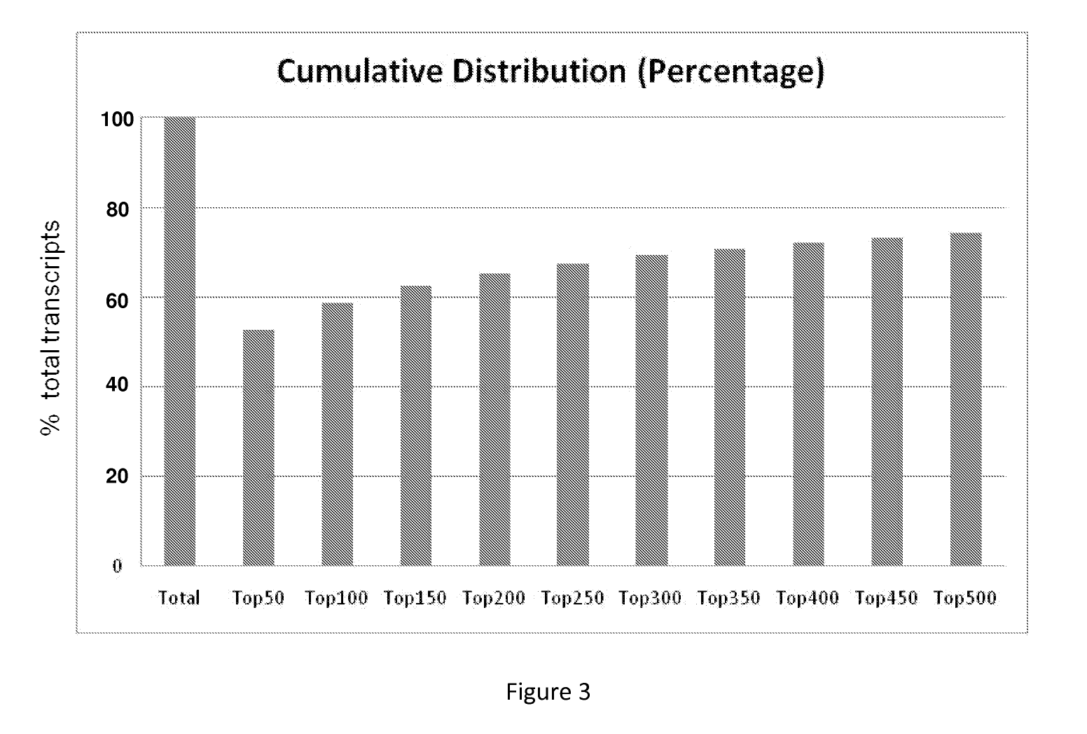

The present invention for removing specific target mRNA includes several steps (FIG. 1). First, the blocker oligonucleotide hybridizes with the target mRNA at the junction region and blocks other oligonucleotides from further hybridizing with the target mRNA at the same junction region. Secondly, the captor oligonucleotides hybridize with the unblocked mRNAs at the junction region, whereas the target mRNA is blocked from binding to the captor oligonucleotides. This step distinguishes the present technology from other technologies such as capturing universal mRNAs with oligo (dT)ₙ (Yeakley et al. 2002). If simple oligo (dT)ₙ is used in our experiment, both blocked and unblocked mRNAs would be captured because mRNAs from mammalian cells have poly(A) tails with approximately 250 nucleotides long. Thirdly, a captor-binding nucleic acid pool is separated from a non-captor-binding nucleic acid pool using capturing methods that are well known to the people skilled in the art. For example, captor oligonucleotides can be linked to biotin and captor-binding nucleic acids can be captured by immobilized streptavidin or avidin. Since the target mRNA is blocked from binding to captor oligonucleotides, an mRNA pool (captor-binding mRNA pool) without target mRNA can then be obtained. Alternatively, reverse transcription can be carried out directly in the reaction during capturing the blocked mRNA pools, and both mRNA and cDNA pools without the target nucleic acid can be captured using biotinylated captor oligonucleotides and streptavidin beads (FIG. 1, left path). The obtained RNA and/or cDNA pools without target genes can be used for further analysis such as high throughput sequencing. Methods to perform reverse transcription are well known to those of ordinary skill in the art and commercial kits such as Superscript VILO cDNA synthesis kit (Invitrogen, Carlsbad, Calif.) are available for performing reverse transcription reactions.

In some embodiments, the present invention further provides methods for retrieving the target nucleic acids. For example, after removing captor-binding nucleic acids from the nucleic acid sample, the target nucleic acids with poly(A) sequence can bind to a poly(dT) column and be eluted from the poly(dT) column. In another embodiment, after captor-binding nucleic acids are removed from the nucleic acid sample, cDNAs of the target nucleic acids can be generated by reverse transcription using the blocker oligonucleotides as primers. cDNAs of the target nucleic acids can then be obtained after RNase H digestion of all the RNAs. In another embodiment, the blocker oligonucleotide may be linked to a poly(C) nucleic acid sequence. The blocker oligonucleotide and associated nucleic acids can be hybridized to a biotinylated poly(G) sequences, which can be further captured by streptavidin-coated magnetic beads.

In particular embodiments of the invention, the blocker oligonucleotide comprises a specific region, which has a sequence complementary to a specific sequence that is adjacent to the 5′-end of poly(A) region of the target nucleic acid, and an oligo(dT/dA) region, wherein the blocker oligonucleotide hybridizes stronger to the target nucleic acid than the captor oligonucleotides. The blocker oligonucleotide, for example, may be complementary to a junction region of the target nucleic acid sequence including the 5′ poly(A) region and the adjacent upstream specific sequence of the target nucleic acid. A key feature of the present invention is that both blocker oligonucleotide and captor oligonucleotides compete for the binding to the junction region of the target nucleic acid. In order for the blocker oligonucleotide to block the binding of the captor oligonucleotide to the junction region of the target nucleic acid, the blocker oligonucleotide is designed to bind stronger to the junction region than the captor oligonucleotides under an appropriate hybridization condition. Nucleic acid analogs such as PNA and LNA have different backbones than that of typical nucleic acids. Given the same sequence, a PNA or a LNA can have higher affinity for the same complimentary nucleic acid sequence than a normal nucleic acid has. PNAs and LNAs are good candidates for using as blocker oligonucleotides. In some embodiments, the blocker oligonucleotide comprises a PNA or a LNA that has a sequence complementary to the junction region of the target nucleic acid. In some embodiments, the blocker oligonucleotide may contain a dideoxynucleotide at the 3′ end of the sequence so that the blocker oligonucleotide is non-extendable in reverse transcription or PCR reactions.

It is understood that the hybridization strength of two strands of nucleic acids depends on the length and nucleotide content of base-pairing nucleotides in the nucleic acid strands. In some embodiments of the invention, the blocker oligonucleotide comprises a specific region that is longer than the specific region of captor oligonucleotides. For example, the specific region of the blocker oligonucleotide is at least 5 bases in length (e.g. at least 5, 6, 7, 8, 9, 10, 11, 12, 13, 14, 15, 16, 17, 18, 19, 20, 21, 22, 23, 24, 25 bases or longer). The specific region of the captor oligonucleotides has at least 1, 2, 3, 4, 5, 6, or more random nucleotides. The blocker oligonucleotides and captor oligonucleotides usually have an oligo(dT/dA) region with the same length of, for example, at least 5, 6, 7, 8, 9, 10, 11, 12, 13, 14, 15, 16, 17, 18, 19, 20 or more nucleotides. In other embodiments of the invention, the blocker oligonucleotide may comprise an oligo(dT/dA) region that is equal or longer than the oligo(dT/dA) region of the captor oligonucleotides. The methods for determining an appropriate condition for a particular hybridization reaction are well known to those of ordinary skill in the art and are described in the public literatures.

In some embodiments, the present invention provides a method for determining the optimal captor oligonucleotides that selectively bind to unblocked mRNAs, comprising: A, making captor oligonucleotides with different number of random nucleotides (e.g. 1, 2, 3, 4, 5, 6 or more random nucleotides) and an oligo(dT/dA) sequence; B, contacting a blocker oligonucleotide specific to an abundant target gene and the captor oligonucleotides to a nucleic acid sample; C, separating the captor-binding nucleic acid pool from the non-captor-binding nucleic acid pool; D, determine the amount of the target gene and an unblocked gene in the captor-binding nucleic acid pool (the blocked pool) and the non-captor-binding nucleic acid pool (the unblocked pool); E, calculate the ratio of the amount of a target gene in the blocked pool vs. unblocked pool (Tᵣₐₜᵢₒ), and the ratio of the amount of an unblocked gene in the blocked pool vs. the unblocked pool (Uᵣₐₜᵢₒ); F, compare the ratio (Finalᵣₐₜᵢₒ) between Tᵣₐₜᵢₒ and Uᵣₐₜᵢₒ for different captor oligonucleotides, wherein the captor oligonucleotide with the lowest Finalᵣₐₜᵢₒ is the optimal captor oligonucleotide. An important aspect of the present invention is to design captor oligonucleotides that selectively bind to unblocked mRNAs but not blocked mRNAs. The optimal captor oligonucleotides will result in highly reduced amount of blocked mRNA in the blocked pool compared to the unblocked pool, reflected in a low value of Tᵣₐₜᵢₒ, and capture most of unblocked mRNAs in the blocked pool, reflected in a high value of Uᵣₐₜᵢₒ. Therefore, an optimal captor oligonucleotide can be chosen based on the lowest value of Finalᵣₐₜᵢₒ (Tᵣₐₜᵢₒ/Uᵣₐₜᵢₒ). The methods to measure the amount of a particular nucleic acid such as southern blot, northern blot, or PCR (Polymerase Chain Reaction) are standard methods in molecular biology and well known to those of ordinary skill in the art.

In some embodiments of the invention, the captor oligonucleotides are linked to a capture structure, which allows the separation of the captor oligonucleotides and any specifically bound nucleic acids from other nucleic acid populations. Methods to separate nucleic acids and their specifically bound compounds are well known to those of ordinary skill in the art. Non-limiting examples of the separation methods include using physical separation, magnetic separation, ligand-receptor binding, antigen-antibody association, or complementary nucleic acid pairing. For example, the captor oligonucleotides may be directly linked to a capture structure such as magnetic beads and be separated from other nucleic acids using a magnetic field. The capture structure of captor oligonucleotides may be a ligand that allows captor-binding nucleic acids to be separated by its receptor coupled to structures such as agarose or magnetic beads. The capture structure may also be an antigen that can be separated by binding to its antibody coupled on structures such as agarose, plastic beads or glass beads. In another example, captor oligonucleotides may be linked to a capture nucleic acid which can bind to its complementary nucleic acid immobilized to magnetic beads. It is contemplated that the capture nucleic acid may be a repeat of G or C residues, or any suitable random sequences. In particular embodiments of the invention, the captor oligonucleotides are covalently linked to a biotin. And the captor-binding nucleic acid pool can be separated by binding to immobilized streptavidin or avidin beads (e.g. streptavidin-coated magnetic beads or avidin-coated magnetic beads). Methods to make a biotinylated nucleic acid are well known to those of ordinary skill in the art (Meier T & Fahrenholz F, 1996). For example, biotin can be linked via a phosphate group to the 5′-end of a captor oligonucleotide or be linked by a suitable linking agent such as a triethylene glycol (TEG) linker. Such biotin labels can be readily prepared from agents such as biotin phosphoramide and biotin TEG phosphoramide. Streptavidin-coated magnetic beads suitable for isolating biotinylated nucleic acids are commercially available from a number of companies such as Sigma-Aldrich (St. Louis, Mo.), Invitrogen (Carlsbad, Calif.), and New England Biolabs (Ipswich, Mass.). Protocols for capturing and eluting biotinylated nucleic acids using the streptavidin magnetic beads can be found at the company's instruction literatures.

In some embodiments of the invention, a plurality of the blocker oligonucleotides are used to hybridize to a plurality of target nucleic acids, blocking them from binding to the captor oligonucleotides. The method can be applied to make a nucleic acid pool with removal of a plurality of target nucleic acids. In some embodiments of the invention, the blocker oligonucleotides are designed to be complementary to specific sequences of high abundance nucleic acids in a transcriptome. The method can be applied to remove a plurality of high abundance nucleic acids from a nucleic acid sample. In particular embodiments of the invention, the blocker oligonucleotides are designed to be complementary to top 10, 50, 200, or 500 genes with highest expression ranking in a particular transcriptome. The high throughput sequence analysis generates hundreds of millions of short sequence reads/tags about nucleic acids in a sample. The abundance of a particular RNA/DNA species can be measured by the number of the generated sequence tags. The expression levels of gene products in a transcriptome can then be ranked based on the high throughput sequence tag numbers. The rankings for gene expression are available for some transcriptomes (Mortazavi et al. 2008), where blocker oligonucleotides against high abundance genes can be designed based on the method described herein. If the ranking data of a particular transcriptome are not available, a high throughput sequence analysis of a total mRNA sample can be performed to generate a ranking list of high abundance genes. The blocker oligonucleotides can be designed against top genes with highest expression ranking based on the results of the sequence analysis (e.g. top 1-50, 50-200, or 200-500 ranking genes). The method of present invention can then be applied to remove the high abundance genes in the transcriptome. As top 500 high abundance genes account for around 80% of total mRNA, reduction of these gene products to low levels can significantly increase the sensitivity and accuracy of measurement of low abundance genes in the transcriptome research.

The present invention provides a simple, cost-effective and flexible method to obtain a nucleic acid pool with the removal of target nucleic acids. This method needs only oligonucleotides (such as blockers and captors) and simple separation methods such as a biotin-streptavidin paired method, but does not need complicated procedures such as enzyme digestion and purification. Since this method uses the corresponding number of blocker oligonucleotides specific for target RNAs and universal biotinylated captor oligonucleotides, its cost is kept low and acceptable. Because the blockers can be common oligonucleotides without any modification, several hundreds of oligonucleotides specific for targets can be made at low costs and it is easy to add/remove blocker oligonucleotides for different target genes. In addition, RNA and/or cDNA pools without target genes can be generated in one experiment for any further analysis. Besides, target RNA or cDNA pools can also be obtained simultaneously if desired.

In some embodiments, present invention provides a composition, comprising: A, a blocker oligonucleotide, wherein the blocker oligonucleotide comprises a specific region, which has a sequence complementary to a specific sequence of a target nucleic acid, and an oligo(dT/dA) region; B, captor oligonucleotides, wherein the captor oligonucleotides comprise a specific region, which has several random nucleotides, and an oligo(dT/dA) region. In particular embodiments, the composition comprises a blocker oligonucleotide that comprises a specific region, which has a sequence complementary to a specific sequence that is adjacent to the 5′-end of poly(A) region of the target nucleic acid. In some embodiments, the composition comprises blocker oligonucleotides that comprise specific regions, which have sequences complementary to top genes with the highest expression ranking in a trascriptome (e.g. top 1-50, 50-200, or 200-500 ranking genes). In some embodiments, the composition comprises captor oligonucleotides that are linked to a capture structure that allows the separation of the captor-binding nucleic acids from other nucleic acid populations. In particular embodiments, the composition comprises captor oligonucleotides linked to biotin. In additional embodiments, composition further comprises immobilized streptavidin (e.g. streptavidin-coated magnetic beads).

In some embodiments, the present invention provides a kit for removing a target nucleic acid from a nucleic acid pool, comprising: A, a blocker oligonucleotide, wherein the blocker oligonucleotide comprises a specific region, which has a sequence complementary to a specific sequence of the target nucleic acid, and an oligo(dT/dA) region; B, captor oligonucleotides, wherein the captor oligonucleotides comprise a specific region, which has several random nucleotides, and an oligo(dT/dA) region.

### EXAMPLES

The following example is provided in order to demonstrate and illustrate certain embodiments and aspects of the present invention, and is not to be construed as limiting the scope of the invention thereof.

### Example 1

### Removal of a Target mRNA from a Total RNA Sample Using a Target-Specific Blocker

The example describes a method to obtain an mRNA pool with removal of a specific target mRNA. The design of the method is to employ a blocker oligonucleotide and universal captor oligonucleotides that are complementary to the junction region of mRNAs (see FIG. 1). Universal captor oligonucleotides have four random nucleotides and a repeat sequence of fifteen dTs. Blocker oligonucleotides have a longer sequence complementary to the specific sequence of the target mRNA and a repeat sequence of fifteen dTs. Since the blocker oligonucleotide has a longer sequence complementary to the junction region of the target mRNA than the captor oligonucleotides, captor oligonucleotides is blocked from binding to the target mRNA in the presence of the blocker oligonucleotide. Thus, the method provides a captor-binding mRNA pool without the target mRNA.

GAPDH, chosen as a target gene here, is a housekeeping gene with a relatively high expression level. The blocker oligonucleotide specific for GAPDH is designed against the junction region of GAPDH gene with 20 specific complementary nucleotides (SEQ ID NO 1) and a (dT)₁₅ sequence, and the biotinylated captor oligonucleotides universal for all mRNAs consists of four random nucleotides and a (dT)₁₅ sequence. When the GAPDH blocker and captor oligonucleotides were mixed with the total RNA sample, the GAPDH blocker oligonucleotide specifically hybridized with GAPDH mRNAs (blocked mRNA) and blocked captor oligonucleotides from hybridizing with GAPDH mRNA. The biotinylated captor oligonucleotides thus selectively hybridized with unblocked mRNAs, and captured unblocked mRNAs to streptavidin-coated magnetic beads. The majority of GAPDH mRNAs remained in the supernatant.

Total RNAs were extracted from LNCaP cultured cells. 1 μg of total RNA, 0.25 μM GAPDH blocker oligonucleotides, 1 μM captor oligonucleotides, 5 μl streptavidin-coated magnetic beads (Invitrogen, Carlsbad, Calif.) and hybridization buffer (50 mM Tris-HCl, 75 mM KCl, 3 mM MgCl₂, where reverse transcription can be directly carried out) were mixed (20 μl reaction volume) in a PCR tube and mounted on PCR machine, which was programmed with 65° C. for 5 min, 60° C. for 5 min, 55° C. for 5 min, 50° C. for 5 min, 45° C. for 5 min and 37° C. for 1 hour. The PCR tube was then mounted on a magnetic stand for 3 min and supernatant was collected. 20 μl H₂O was added into the tube with magnetic beads only and PCR tube was heated at 65° C. for 5 min. Again, the PCR tube was mounted on magnetic stand for 3 min and the eluate was collected. The supernatant (GAPDH-rich pool) and the eluate from magnetic beads (GAPDH-absent pool) were reverse transcribed with oligo (dT)₁₅ to prepare cDNA. Using two PCR primer pairs for GAPDH and KLK3, two genes were amplified with 23 PCR cycles (Invitrogen, Carlsbad, Calif.). PCR products were separated on a 2% agarose gel stained with ethidium bromide. Note that most of GAPDH (blocked mRNA) was left in the supernatant and much less was present in the eluate from the streptavidin beads. On the contrary, most of KLK3 (unblocked mRNA) was enriched on the streptavidin beads and much less was left in the supernatant (see FIG. 2). The results clearly showed that a GAPDH-absent mRNA pool was created using the method of present invention.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

### REFERENCES

Alberts, B. et al. (1994) Molecular biology of the cell, 3^{rd} ed., New York: Garland Publishing, Inc
Allison, D., Cui, X., Page, G., and Sabripour, M. Microarray data analysis: From disarray to consolidation and consensus. Nat. Rev. Genet. 2006; 7: 55-65.
Bennett, S., Barnes, C., Cox, A., Davies, L., and Brown, C. Toward the 1,000 dollars human genome. Pharmacogenomics 2005; 6: 373-382.
Cokus, S., Feng, S., Zhang, X., Chen, Z., Merriman, B., Haudenschild, C., Pradhan, S., Nelson, S., Pellegrini, M., and Jacobsen, S. Shotgun bisulphite sequencing of the Arabidopsis genome reveals DNA methylation patterning. Nature 2008; 452: 215-219.
Dahl F, Stenberg J, Fredriksson S, Welch K, Zhang M, Nilsson M, Bicknell D, Bodmer W F, Davis R W, Ji H. Multigene amplification and massively parallel sequencing for cancer mutation discovery. Proc Natl Acad Sci USA. 2007; 104(22):9387-92.
Fu X, Fu N, Guo S, Yan Z, Xu Y, Hu H, Menzel C, Chen W, Li Y, Zeng R, Khaitovich P. Estimating accuracy of RNA-Seq and microarrays with proteomics. BMC Genomics. 2009; 10:161.

Gautier, L., Cope, L., Bolstad, B., and Irizarry, R. affy—Analysis of Affymetrix GeneChip data at the probe level. Bioinformatics 2004; 20: 307-315.
Gnirke A, Melnikov A, Maguire J, Rogov P, LeProust E M, Brockman W, Fennell T, Giannoukos G, Fisher S, Russ C, Gabriel S, Jaffe D B, Lander E S, Nusbaum C. Solution hybrid selection with ultra-long oligonucleotides for massively parallel targeted sequencing. Nat Biotechnol. 2009; 27(2):182-9
Harris T D, Buzby P R, Babcock H, Beer E, Bowers J, Braslaysky I, Causey M, Colonell J, Dimeo J, Efcavitch J W, Giladi E, Gill J, Healy J, Jarosz M, Lapen D, Moulton K, Quake S R, Steinmann K, Thayer E, Tyurina A, Ward R, Weiss H, Xie Z.Single-molecule DNA sequencing of a viral genome. Science. 2008; 320(5872):106-9.
Korbel, J., Urban, A., Affourtit, J., Godwin, B., Grubert, F., Simons, J., Kim, P., Palejev, D., Carriero, N., Du, L., et al. Paired-end mapping reveals extensive structural variation in the human genome. Science 2007; 318: 420-426.
Margulies, M., Egholm, M., Altman, W., Attiya, S., Bader, J., Bemben, L., Berka, J., Braverman, M., Chen, Y., Chen, Z., et al. Genome sequencing in microfabricated high-density picolitre reactors. Nature 2005; 437: 376-380.
Marioni J C, Mason C E, Mane S M, Stephens M, Gilad Y. RNA-seq: an assessment of technical reproducibility and comparison with gene expression arrays. Genome Res. 2008; 18(9):1509-17.
Meier T & Fahrenholz F (Eds.) A Laboratory Guide to Biotin-Labeling in Biomolecule Analysis (Biomethods), Spinger-Verlag Telos, 1996.
Mikkelsen, T., Ku, M., Jaffe, D., Issac, B., Lieberman, E., Giannoukos, G., Alvarez, P., Brockman, W., Kim, T., Koche, R., et al. Genome-wide maps of chromatin state in pluripotent and lineage-committed cells. Nature 2007;448: 553-560.
Mortazavi A, Williams B A, McCue K, Schaeffer L, Wold B. Mapping and quantifying mammalian transcriptomes by RNA-Seq. Nat Methods. 2008; 5(7):621-8
Okou D T, Steinberg K M, Middle C, Cutler D J, Albert T J, Zwick M E. Microarray-based genomic selection for high-throughput resequencing. Nat Methods. 2007; 4(11):907-9.
Passador-Gurgel, G., Hsieh, W., Hunt, P., Deighton, N., and Gibson, G. Quantitative trait transcripts for nicotine resistance in Drosophila melanogaster. Nat. Genet. 2007;39: 264-268.
Porreca G J, Zhang K, Li J B, Xie B, Austin D, Vassallo S L, LeProust E M, Peck B J, Emig C J, Dahl F, Gao Y, Church G M, Shendure J.Multiplex amplification of large sets of human exons. Nat Methods. 2007; 4(11):931-6.
Rifkin, S., Kim, J., and White, K. Evolution of gene expression in the Drosophila melanogaster subgroup. Nat. Genet. 2003; 33: 138-144.
Scherf, U., Ross, D. T., Waltham, M., Smith, L. H., Lee, J. K., Tanabe, L., Kohn, K. W., Reinhold, W. C., Myers, T. G., Andrews, D. T. et al. A gene expression database for the molecular pharmacology of cancer. Nat. Genet. 2000; 24: 236-244.
Tang F, Barbacioru C, Wang Y, Nordman E, Lee C, Xu N, Wang X, Bodeau J, Tuch B B, Siddiqui A, Lao K, Surani M A. mRNA-Seq whole-transcriptome analysis of a single cell. Nat Methods. 2009; 6(5):377-82
White, K. Functional genomics and the study of development, variation and evolution. Nat. Rev. Genet. 2001; 2: 528-537.
Yeakley J M, Fan J B, Doucet D, Luo L, Wickham E, Ye Z, Chee M S, Fu X D. Profiling alternative splicing on fiber-optic arrays. Nat Biotechnol. 2002; 20(4):353-8.

What is claim is:

1. A method of obtaining a nucleic acid pool with removal of a target nucleic acid from a nucleic acid sample, comprising steps of:
a, contacting said nucleic acid sample with a blocker oligonucleotide, wherein said blocker oligonucleotide comprises a specific region and a oligo(dT/dA) region;
b, contacting said nucleic acid sample with captor oligonucleotides, wherein said captor oligonucleotides comprise a specific region, which has random nucleotides, and a oligo(dT/dA) region;
c, separating a captor-binding nucleic acid pool from a nucleic acid pool that does not hybridize to said captor oligonucleotides, therefore obtaining said nucleic acid pool with no or reduced amount of said target nucleic acid.

2. The method of claim 1, wherein said specific region of said blocker oligonucleotide comprises a sequence complementary to a specific sequence adjacent to the 5′-end of poly(A) region of said target nucleic acid.

3. The method of claim 1, wherein said blocker oligonucleotide comprises a PNA, LNA or 3′ dideoxynucleotide.

4. The method of claim 1, wherein said blocker oligonucleotide further comprises a poly(C) or poly(G) nucleotide sequence.

5. The method of claim 1 further comprising a reverse transcription reaction to synthesize cDNAs from an mRNA sample.

6. The method of claim 1, wherein said specific region of said blocker oligonucleotide is at least 5 bases in length.

7. The method of claim 1, wherein a plurality of said blocker oligonucleotides are used to remove a plurality of target nucleic acids.

8. The method of claim 7, wherein said blocker oligonucleotides are designed to hybridize to top 1-10, 10-50, 50-200, or 200-500 genes with highest expression ranking in a transcriptome.

9. The method of claim 8, wherein said blocker oligonucleotides are designed to hybridize to top 50 highest expression genes.

10. The method of claim 8, wherein said blocker oligonucleotides are designed to hybridize to top 200 highest expression genes.

11. The method of claim 8, wherein said blocker oligonucleotides are designed to hybridize to top 500 highest expression genes.

12. The method of claim 1, wherein said captor oligonucleotides are linked to a capture structure.

13. The method of claim 12, wherein said capture structure of said captor oligonucleotides is a biotin.

14. The method of claim 13, wherein said captor-binding nucleic acid pool is separated by binding to immobilized streptavidin.

15. A composition for removing a target nucleic acid from a nucleic acid sample, comprising:
a, a blocker oligonucleotide, wherein said blocker oligonucleotide comprises a specific region and a oligo(dT/dA) region;
b, captor oligonucleotides, wherein said captor oligonucleotides comprise a specific region with random nucleotides and a oligo(dT/dA) region.

16. A composition of claim 15, wherein said specific region of said blocker oligonucleotide is complementary to a specific sequence adjacent to the 5′-end of poly(A) region of said target nucleic acid.

17. A composition of claim 15, wherein said blocker oligonucleotides comprise specific regions complementary to top 10-50, 50-200, or 200-500 highest expression genes in a transcriptome.

18. A composition of claim 15, wherein said captor oligonucleotides are linked to biotin.

19. A composition of claim 18, further comprises immobilized streptavidin.

20. A kit for removing a target nucleic acid from a nucleic acid sample, comprising:
a, a blocker oligonucleotide, wherein said blocker oligonucleotide comprises a specific region, which is complementary to a specific sequence of said target nucleic acid, and a oligo(dT/dA) region;
b, captor oligonucleotides, wherein said captor oligonucleotides comprise a specific region with random nucleotides and a oligo(dT/dA) region.


SEQUENCE LISTING (1 sequence)

SEQ ID NO: 1 - DNA, 20 bp, Homo Sapiens
  Synthetic GAPDH blocker
ggttgagcacagggtacttt